(19) United States
(12) Patent Application Publication
(10) Pub. No.: US 2014/0088637 A1
(43) Pub. Date: Mar. 27, 2014

(54) Magnetic Collet for Attaching End Effector

(71) Applicant: ETHICON ENDO-SURGERY, INC., Cincinnati, OH (US)

(72) Inventors: Shailendra K. Parihar, Mason, OH (US); Patrick J. Minnelli, Harrison, OH (US); Foster B. Stulen, Mason, OH (US)

(73) Assignee: Ethicon Endo-Surgery, Inc., Cincinnati, OH (US)

(21) Appl. No.: 13/627,211

(22) Filed: Sep. 26, 2012

(51) Int. Cl. A61B 17/29

(52) U.S. Cl. 606/205

(57) ABSTRACT

A surgical device comprises an elongate shaft comprising an outer tube and an inner rod positioned in the outer tube, the outer tube and inner rod each comprising a distal end and a proximal end. An actuator is operably connected to the proximal ends of the outer tube and the inner rod. An end effector is adapted for in vivo attachment to and detachment from the elongate shaft. The end effector comprises an outer casing comprising a mating feature adapted to attach to the distal end of the outer tube; an inner shuttle axially moveable relative the outer casing, the shuttle comprising a magnetic material providing provisional engagement to the distal end of the inner rod; a pair of surgical jaws having an opened position and closed position dependant on the relative axial position of the shuttle and casing. The surgical device may further comprise a collet on the distal end of the inner rod adapted to lock onto the shuttle.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While a variety of different minimally invasive surgical devices are known, no one has previously made or used the surgical devices and methods in accordance with the present invention

### SUMMARY

In one embodiment, a surgical device comprises an elongate shaft comprising an outer tube and an inner rod positioned in the outer tube, the outer tube and inner rod each comprising a distal end and a proximal end. An actuator is operably connected to the proximal ends of the outer tube and the inner rod. A collet is on the distal end of the inner rod, the collet having a locked position and an unlocked position dependant on the axial position of the inner rod relative to the outer tube. An end effector is adapted for in vivo attachment to and detachment from the elongate shaft. The end effector comprises an outer casing comprising a mating feature adapted to attach to the distal end of the outer tube; an inner shuttle axially moveable relative the outer casing, the shuttle comprising a proximal end sized and dimensioned to be received in and locked to the collet; and a pair of surgical jaws having an opened position and closed position dependant on the relative axial position of the shuttle and casing.

The proximal tip of the end effector shuttle may comprises a bulbous feature. The bulbous feature and collet may include materials facilitating a magnetic engagement to one another. The bulbous feature may comprise at least a portion of a sphere. The collet may comprise a plurality of arms each with a medially oriented tooth. The shuttle may comprise a notch dimensioned to receive the teeth. The actuator may comprise a manual handle. The collet may comprise a plurality of laterally biased arms. Proximal motion of the inner rod relative the outer tube may cause the outer tube to medially deflect the collet arms. The shuttle and collet may include materials facilitating a magnetic engagement to one another.

In another embodiment, a surgical device comprises an elongate shaft having an outer tube and an inner rod positioned in the outer tube, the outer tube and inner rod each comprising a distal end and a proximal end. An actuator is operably connected to the proximal ends of the outer tube and the inner rod. An end effector is adapted for in vivo attachment to and detachment from the distal ends of the outer tube and the inner rod. A means is provided for locking engagement between the distal end of the inner rod and the end effector. The surgical device may further comprise a means for locking engagement between the distal end of the outer tube and the end effector.

In yet another embodiment, a surgical device comprises an elongate shaft comprising an outer tube and an inner rod positioned in the outer tube, the outer tube and inner rod each comprising a distal end and a proximal end. An actuator is operably connected to the proximal ends of the outer tube and the inner rod. An end effector is adapted for in vivo attachment to and detachment from the elongate shaft. The end effector comprises an outer casing comprising a mating feature adapted to attach to the distal end of the outer tube; an inner shuttle axially moveable relative the outer casing, the shuttle comprising a magnetic material providing provisional engagement to the distal end of the inner rod; a pair of surgical jaws having an opened position and closed position dependant on the relative axial position of the shuttle and casing. The surgical device may further comprise a collet on the distal end of the inner rod adapted to lock onto the shuttle.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts an example of an end effector;

FIG. 2 depicts an example of an elongate shaft;

FIG. 3 depicts an example of an actuator;

FIG. 4 depicts the end effector of FIG. 1 and the elongate shaft if FIG. 2;

FIG. 5 depicts the end effector and elongate shaft connected to one another;

FIG. 6 depicts an exploded view of the end effector and elongate shaft;

FIG. 7 depicts an cross-sectional view of the end effector and elongate shaft with the collet in the unlocked position; and

FIG. 8 depicts an cross-sectional view of the end effector and elongate shaft connected to one another with the collet in the locked position.

### DETAILED DESCRIPTION

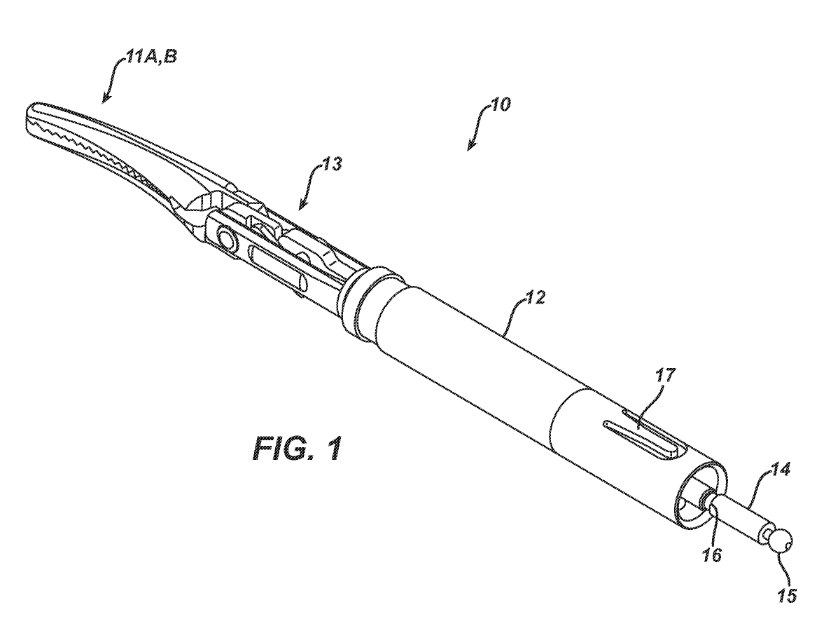

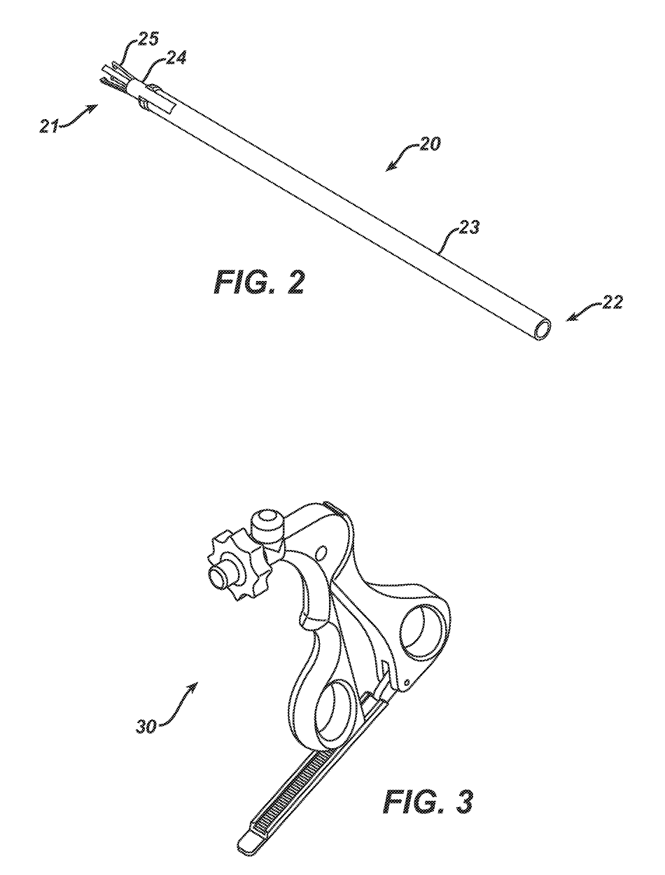

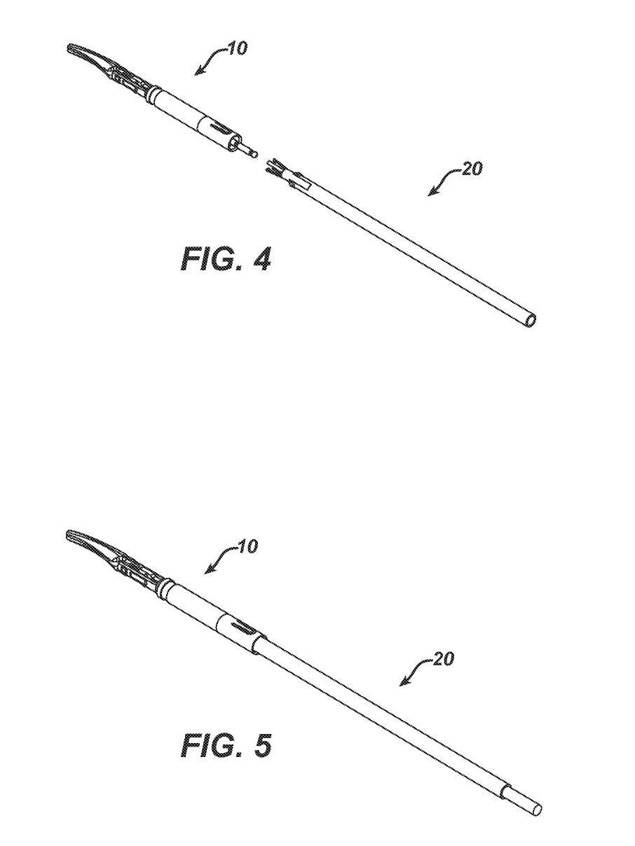

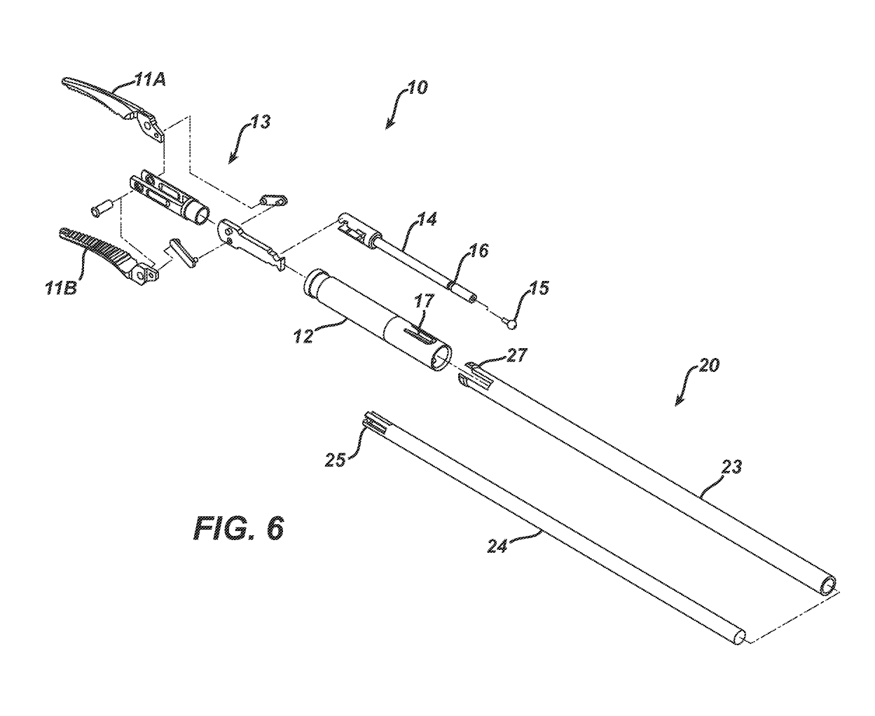

FIGS. 1-5 illustrate one example a laparoscopic surgical instrument. The elongate shaft (20) comprises an outer tube (23) and an inner rod (24) positioned in the outer tube (23). The elongate shaft comprises a distal end (21) and a proximal end (22). The elongate shaft (20) may be rigid and adapted for insertion into a body cavity through an access device, such a trocar, or through direct percutaneous insertion without an access device. The elongate shaft (20) may also be flexible and sized for insertion through the working channel of a flexible endoscope. The rigid shaft embodiment is well suited for laparoscopic surgical procedures, and the flexible shaft embodiment is well suited for natural orifice intraluminal and natural orifice transluminal procedures.

An actuator (30) is operably connected to the proximal ends of the outer tube (23) and the inner rod (24). The actuator (30) causes the inner rod (24) to move relative the outer tube (23). In this embodiment the actuator (30) is a manual pistol grip handle; however, a variety of other manual actuators could also be used, including a scissor grip handle, a syringe grip handle, endoscopic rotary knobs, and the like. The actuator (30) could also take the form of a robotic interface, such as an DAVINCI puck, a housing comprising gears or pulleys, servomechanisms, and the like.

A collet (25) is positioned on the distal end of the inner rod (24). In this embodiment the collet (25) comprises a plurality of arms each with a medially oriented tooth. As shown here, four arms are embodied, but more or fewer arms could also work. Each arm is biased laterally outward thus flaring the collet (25) open. The opened arms corresponds to the unlock position. Pulling the inner rod (24) proximally relative the outer tube (23) will draw the arms into the outer tube (23) thus forcing the arms to deflect medially and closing the collet (25). The closed arms corresponds to the locked position. Thus, collet (25) has a locked position and an unlocked position dependant on the axial position of the inner rod (24) relative to the outer tube (23).

The end effector (10) is adapted for in vivo attachment to and detachment from the elongate shaft (20). The end effector (10) comprises a mating feature (17) adapted to attach the outer casing (12) to the distal end of the outer tube (23). In this embodiment the mating feature (17) is a detent-type mechanism comprising two leaf springs, positioned 180 degrees from each other on the outer casing (12), each having a medial facing tooth. The outer tube (23) slides distally into the outer casing (12), and the leaf springs provide a biased snap-to-lock engagement of the teeth over and past the lip (27). Other mating features could also be used, including threads, collets, bayonets, and the like.

An inner shuttle (14) is axially moveable relative the outer casing (12). The shuttle comprises a proximal end sized and dimensioned to be received in and locked to the collet (25). In this embodiment, the shuttle (14) extends proximally from the outer casing (12) and comprises a notch (16) dimensioned to receive the collet (25) teeth in the locked position.

A pair of surgical jaws (11A, B) have an opened position and closed position dependant on the relative axial position of the shuttle (14) and the outer casing (12). A bar linkage and clevis mechanism (13) translates the axial motion of the shuttle (14) to open and close the jaws (11); however, a variety of other known mechanisms can be used to effect operation of the jaws (11). In this embodiment, the jaws (11) are shown as a dissector; however, a variety of other tissue manipulating jaws could also be used, including graspers, sheers, babcocks, forceps, staplers, clip appliers, and the like. Non-jawed end effectors could also be employed such as hook knives, snares, retractors, and the like. In the case of end effectors that require energy, appropriate energy transmission mechanisms known in the art can be added. For instance, appropriate electrical connections can be added between the shaft (20) and end effector (10) to enable bi-polar forceps. Similarly, an ultrasonic transducer and waveguide can be added for the ultrasonic shears end effector.

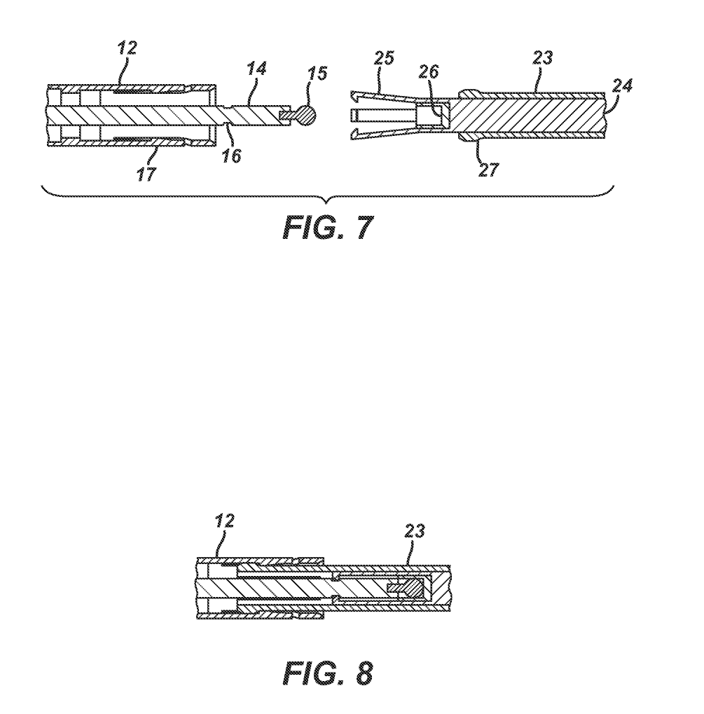

FIGS. 7-8 illustrate further details of how the end effector (10) attaches to the shaft (20). The shuttle (14) and collet (25) may include materials to facilitate a provisional magnetic engagement to one another. In this embodiment, the proximal tip of the shuttle (14) comprising a bulbous feature (15), shown here as partially spherical, formed from a magnetic material. The collet (25) includes a seat (26) made from a material attracted to magnets. Close approximation of the end effector (10) to the distal end (21) of the shaft (20) will cause the shuttle (14) to jump into the seat (26) by virtue of the magnetic attraction. The magnetic facilitates both initial alignment and engagement of the end effector (10) to the shaft (20), but also facilitates the shuttle (14) being fully seated into the collet (25) prior to locking.

During surgery, the distal end (21) of the shaft (20) is inserted into the body cavity, such as the abdomen, pelvis, thorax, etc. The end effector (10) as also introduced into the body cavity, typically through an access port. Optionally, the end effector (10) can be introduced with a separate loader, such as that disclosed in U.S. application Ser. No. 12/576,565. The shuttle (14) is then seated into the collet (25), which may be facilitated by the shuttle (14) and collet (25) having magnetic attraction to one another. After the shuttle (14) is seated into the collet (25), the actuator (30) can be used to pull the inner rod (24) proximally relative the outer tube (23) to close and lock the collet (25) onto the shuttle (14). Continued proximate pulling of the inner rod (24) will cause the outer tube (23) to be inserted into the outer casing (12) until the mating feature (17) is engaged and locked to the outer tube (23). Thus, the end effector (10) is now fully attached in vivo to the shaft (20). Operation of the actuator (30) translates to operate the jaws (11), thus enabling a surgeon to manipulate tissue and perform minimally invasive surgical procedures.

In vivo detachment is achieved by reversing the attachment steps outlined in the previous paragraph. Using the actuator (30), the outer tube (23) is pulled proximally relative the inner rod (24) until the outer tube (23) disengages from the mating feature (17) and is withdrawn from the outer casing (12). Continued proximal pulling of the outer tube (23) will then open and unlock the collet (25). The end effector (10) can then be withdrawn from the collet (25). Preferably, any magnetic attraction forces between the shuttle (14) and collet (25) would be sufficiently low as to make separation simple.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical device, comprising:
a) an elongate shaft comprising an outer tube and an inner rod positioned in the outer tube, the outer tube and inner rod each comprising a distal end and a proximal end;
b) an actuator operably connected to the proximal ends of the outer tube and the inner rod;
c) a collet on the distal end of the inner rod, the collet having a locked position and an unlocked position dependant on the axial position of the inner rod relative to the outer tube;
d) an end effector adapted for in vivo attachment to and detachment from the elongate shaft, the end effector comprising:
i) an outer casing comprising a mating feature adapted to attach to the distal end of the outer tube;
ii) an inner shuttle axially moveable relative the outer casing, the shuttle comprising a proximal end sized and dimensioned to be received in and locked to the collet;
iii) a pair of surgical jaws having an opened position and closed position dependant on the relative axial position of the shuttle and casing.

2. The surgical device of claim 1 wherein the proximal tip of the end effector shuttle comprising a bulbous feature.

3. The surgical device of claim 2, wherein the bulbous feature and collet include materials facilitating a magnetic engagement to one another.

4. The surgical device of claim 2, wherein the bulbous feature comprises at least a portion of a sphere.

5. The surgical device of claim 1, wherein the collet comprises a plurality of arms each with a medially oriented tooth.

6. The surgical device of claim 5, wherein the end effector shuttle comprises a notch dimensioned to receive the teeth.

7. The surgical device of claim 1, wherein the actuator comprises a manual handle.

8. The surgical device of claim 1, wherein the collet comprises a plurality of laterally biased arms.

9. The surgical device of claim 8, wherein proximal motion of the inner rod relative the outer tube causes the outer tube to medially deflect the collet arms.

10. The surgical device of claim 1, wherein the shuttle and collet include materials facilitating a magnetic engagement to one another.

11. A surgical device, comprising:
an elongate shaft comprising an outer tube and an inner rod positioned in the outer tube, the outer tube and inner rod each comprising a distal end and a proximal end;
an actuator operably connected to the proximal ends of the outer tube and the inner rod;
an end effector adapted for in vivo attachment to and detachment from the distal ends of the outer tube and the inner rod;
a means for locking engagement between the distal end of the inner rod and the end effector.

12. The surgical device of claim 11, further comprising a means for locking engagement between the distal end of the outer tube and the end effector.

13. A surgical device, comprising:
a) an elongate shaft comprising an outer tube and an inner rod positioned in the outer tube, the outer tube and inner rod each comprising a distal end and a proximal end;
b) an actuator operably connected to the proximal ends of the outer tube and the inner rod;
c) an end effector adapted for in vivo attachment to and detachment from the elongate shaft, the end effector comprising:
i) an outer casing comprising a mating feature adapted to attach to the distal end of the outer tube;
ii) an inner shuttle axially moveable relative the outer casing, the shuttle comprising a magnetic material providing provisional engagement to the distal end of the inner rod;
iii) a pair of surgical jaws having an opened position and closed position dependant on the relative axial position of the shuttle and casing.

14. The surgical device of claim 13, further comprising a collet on the distal end of the inner rod adapted to lock onto the shuttle.
